(19) United States
(12) Patent Application Publication
(10) Pub. No.: US 2025/0309603 A1
(43) Pub. Date: Oct. 2, 2025

(54) DUAL WAVELENGTH OPTICAL PULSE GENERATOR

(71) Applicant: UNIVERSITY OF SEOUL INDUSTRY COOPERATION FOUNDATION, Seoul (KR)

(72) Inventors: Ju Han LEE, Seoul (KR); Jee Hwan KIM, Seoul (KR)

(73) Assignee: UNIVERSITY OF SEOUL INDUSTRY COOPERATION FOUNDATION, Seoul (KR)

(21) Appl. No.: 19/074,866

(22) Filed: Mar. 10, 2025

(30) Foreign Application Priority Data
Mar. 28, 2024 (KR) 10-2024-0042322

(51) Int. Cl. H01S 3/08; H01S 3/067; H01S 3/16

(52) U.S. Cl. CPC H01S 3/0809; H01S 3/06716; H01S 3/0675; H01S 3/08013; H01S 3/1616

(57) ABSTRACT

The present invention provides a dual wavelength optical pulse generator capable of generating an optical pulse dual wavelengths using a thulium-doped optical fiber. A dual wavelength optical pulse generator according to the present invention includes: an optical pump generating a first optical pulse having a first wavelength; an optical resonator generating a second optical pulse having a second wavelength from the first optical pulse generated by the optical pump; a first optical wavelength division multiplexer/demultiplexer supplying the first optical pulse generated by the optical pump to a first end of the optical resonator and separating and outputting the second optical pulse generated by the optical resonator; a delay optical fiber delaying the first optical pulse passed through the optical resonator; and a second optical wavelength division multiplexer/demultiplexer combining: the first optical pulse delayed by the delay optical fiber; and the second optical pulse separated and outputted by the first optical wavelength division multiplexer/demultiplexer to output a dual wavelength optical pulse, wherein the optical resonator comprises: a thulium-doped optical fiber generating the second optical pulse from the first optical pulse; a first fiber Bragg grating provided at a first end of the thulium-doped optical fiber and reflecting the second optical pulse outputted from the first end of the thulium-doped optical fiber; and a second fiber Bragg grating provided at a second end of the thulium-doped optical fiber, the second fiber Bragg grating transmitting the first optical pulse therethrough and reflecting the second optical pulse outputted from the second end of the thulium-doped optical fiber.

### CROSS-REFERENCE TO RELATED PATENT APPLICATION

This non-provisional U.S. patent application claims priority under 35 U.S.C. § 119 of Korean Patent Application No. 10-2024-0042322 filed on Mar. 28, 2024, in the KIPO, the entire contents of which are hereby incorporated by reference.

### BACKGROUND OF THE INVENTION

### 1. Field

The present invention relates to a dual wavelength optical pulse generator capable of generating an optical pulse having two wavelengths using a thulium-doped optical fiber. The present invention was derived as a result of researches sponsored by Ministry of Science and ICT, titled “Development of Erbium-doped ZBLAN Fiber-based Lasers Operating at 2.8 μm Wavelengths” (Project No. 1711180749) and “Color-Modulated Hyper-Sensory Perception Technology Research Center” (Project No. 1711199680).

### 2. Description of the Related Art

Recently, research on medical lasers has been actively conducted. Medical lasers are used for the diagnosis and treatment of diseases. For example, laser imaging techniques are used to detect symptoms of diseases or cancerous tissues.

Depending on the equipment used and the target, lasers with a wavelength of 1500 nm band and lasers with a wavelength of 1700 nm band may be used as medical lasers.

Currently, when the bands of the lasers used are different, for example, when both a laser beam with a wavelength of 1500 nm band and a laser beam with a wavelength of 1700 nm band are needed, it is problematic that two separate equipments must be used.

Therefore, the need for medical equipment that is capable of simultaneously generating and selectively providing two lasers with different wavelengths using a single laser generator is emerging.

### SUMMARY OF THE INVENTION

It is an object of the present invention to provide a dual wavelength optical pulse generator capable of generating an optical pulse with dual wavelengths using a thulium-doped optical fiber.

A dual wavelength optical pulse generator according to a first embodiment of the present invention includes: an optical pump generating a first optical pulse having a first wavelength; an optical resonator generating a second optical pulse having a second wavelength from the first optical pulse generated by the optical pump; a first optical wavelength division multiplexer/demultiplexer supplying the first optical pulse generated by the optical pump to a first end of the optical resonator and separating and outputting the second optical pulse generated by the optical resonator; a delay optical fiber delaying the first optical pulse passed through the optical resonator; and a second optical wavelength division multiplexer/demultiplexer combining: the first optical pulse delayed by the delay optical fiber; and the second optical pulse separated and outputted by the first optical wavelength division multiplexer/demultiplexer to output a dual wavelength optical pulse, wherein the optical resonator comprises: a thulium-doped optical fiber generating the second optical pulse from the first optical pulse; a first fiber Bragg grating provided at a first end of the thulium-doped optical fiber and reflecting the second optical pulse outputted from the first end of the thulium-doped optical fiber; and a second fiber Bragg grating provided at a second end of the thulium-doped optical fiber, the second fiber Bragg grating transmitting the first optical pulse therethrough and reflecting the second optical pulse outputted from the second end of the thulium-doped optical fiber.

It is preferable that ranges of the first wavelength and the second wavelength are 1560±30 nm and 1705±30 nm, respectively.

It is preferable that the first fiber Bragg grating reflects 60% to 90% and transmits 10% to 40% of the second optical pulse, respectively.

It is preferable that the second fiber Bragg grating reflects 99% to 100% to 100% and transmits 0% to 0% to 1% of the second optical pulse.

It is preferable that the delay optical fiber includes a single mode optical fiber.

A dual wavelength optical pulse generator according to a second embodiment of the present invention includes: an optical pump generating a first optical pulse having a first wavelength; an optical resonator generating a second optical pulse having a second wavelength from the first optical pulse generated by the optical pump; a optical wavelength division multiplexer/demultiplexer supplying the first optical pulse generated by the optical pump to a first end of the optical resonator and separating and outputting the second optical pulse generated by the optical resonator; a delay optical fiber delaying the first optical pulse passed through the optical resonator; and an optical switch selectively outputting: the first optical pulse passed through the optical resonator; and the second optical pulse separated and outputted by the optical wavelength division multiplexer/demultiplexer, wherein the optical resonator comprises: a thulium-doped optical fiber generating the second optical pulse from the first optical pulse; a first fiber Bragg grating provided at a first end of the thulium-doped optical fiber and reflecting the second optical pulse outputted from the first end of the thulium-doped optical fiber; and a second fiber Bragg grating provided at a second end of the thulium-doped optical fiber, the second fiber Bragg grating transmitting the first optical pulse therethrough and reflecting the second optical pulse outputted from the second end of the thulium-doped optical fiber.

It is preferable that ranges of the first wavelength and the second wavelength are 1560±30 nm and 1705±30 nm, respectively.

It is preferable that the first fiber Bragg grating reflects 60% to 90% and transmits 10% to 40% of the second optical pulse, respectively.

It is preferable that the second fiber Bragg grating reflects 99% to 100% and transmits 0% to 1% of the second optical pulse.

### BRIEF DESCRIPTION OF DRAWINGS

FIG. 1 schematically illustrates a dual wavelength optical pulse generator according to a first embodiment of the present invention.

FIG. 2 schematically illustrates a dual wavelength optical pulse outputted by the dual wavelength optical pulse generator according to the first embodiment of the present invention.

FIG. 3 schematically illustrates a dual wavelength optical pulse generator according to a second embodiment of the present invention.

### DETAILED DESCRIPTION OF THE INVENTION

Hereinafter, a dual wavelength optical pulse generator according to the present invention will be described in detail with reference to the accompanying drawings.

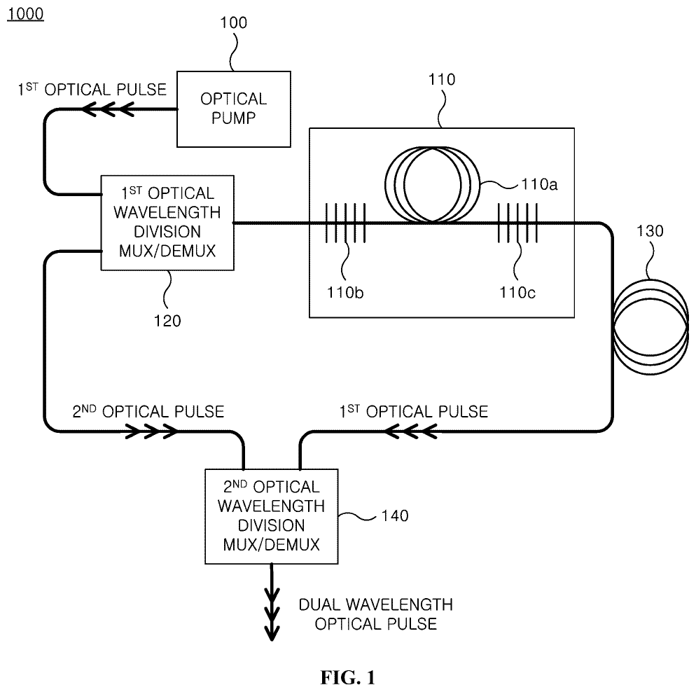

FIG. 1 schematically illustrates a dual wavelength optical pulse generator according to a first embodiment of the present invention.

Referring to FIG. 1, a dual wavelength optical pulse generator 1000 according to a first embodiment of the present invention includes an optical pump 100, an optical resonator 110, a first optical wavelength division multiplexer/demultiplexer 120, a delay optical fiber 130 and a second optical wavelength division multiplexer/demultiplexer 140.

The optical pump 100 generates a first optical pulse (e.g., a pulse laser) having a first wavelength and provides the same to the first optical wavelength division multiplexer/demultiplexer 120.

The optical pump 100 generates a high-power first optical pulse using an erbium-doped optical fiber, a ytterbium-doped optical fiber, etc.,

Preferably, the range of the first wavelength is 1560±30 nm, but is not limited thereto.

The first optical wavelength division multiplexer/demultiplexer 120 provides the first optical pulse generated by the optical pump 100 to a first end of the optical resonator 110, and separates and outputs the second optical pulse generated by the optical resonator 110.

Here, the first optical wavelength division multiplexer/demultiplexer 120 is a bi-directional device. Specifically, when two beams with two different wavelengths, respectively, are inputted, the two beams are combined and outputted as one beam with two wavelengths, and when one beam with two wavelengths is inputted, the one beam is separated and outputted as two beams with two different wavelengths, respectively (a. k. a WDM: wavelength-division multiplexing).

For example, in FIG. 1, the first optical wavelength division multiplexer/demultiplexer 120 may supply the first optical pulse outputted from the optical pump 100 to the optical resonator 110, and also separate the second optical pulse outputted from the optical resonator 110 and supply the separated second optical pulse to the second optical wavelength division multiplexer/demultiplexer 140.

Hereinafter, the “optical wavelength division multiplexer/demultiplexer” refers to a device that can operate bi-directionally to couple or separate beam(s).

The optical resonator 110 generates a second optical pulse having a second wavelength from the first optical pulse generated by the optical pump 100.

Specifically, the optical resonator 110 may include a thulium-doped optical fiber 110a, a first fiber Bragg grating 110b and a second fiber Bragg grating 110c.

The thulium-doped optical fiber 110a generates the second optical pulse having a second wavelength from the first optical pulse generated by the optical pump 100.

The thulium-doped optical fiber 110a generates a second optical pulse having a second wavelength from the first optical pulse having the first wavelength via spontaneous emission and subsequent stimulated emission. That is, the thulium-doped optical fiber 110a acts as a gain medium of a resonator for generating the second optical pulse, which will be described later.

Preferably, the range of the second wavelength is 1705±30 nm, but is not limited thereto.

The first fiber Bragg grating 110b is provided between the first optical wavelength division multiplexer/demultiplexer 120 and the first end of the thulium-doped optical fiber 110a to partially reflect the second optical pulse outputted from the first end of the thulium-doped optical fiber 110a. The first fiber Bragg grating 110b functions as a mirror that constitutes the optical resonator 110.

The first fiber Bragg grating 110b may reflect a portion of the second optical pulse outputted from the first end of the thulium-doped optical fiber 110a while transmitting the remaining portion.

For example, the first fiber Bragg grating 110b may reflect 60% to 90% and transmits 10% to 40% of the second optical pulse, respectively.

The second fiber Bragg grating 110c is provided at a second end of the thulium-doped optical fiber 110a and transmits the first optical pulse outputted from the second end of the thulium-doped optical fiber 110a and also reflects the second optical pulse.

Specifically, the second fiber Bragg grating 110c may transmit the entirety of the first optical pulse remaining after being used as a pump beam. The second fiber Bragg grating 110c functions as a mirror constituting the optical resonator 110.

The second fiber Bragg grating 110c may reflect a portion of the second optical pulse outputted from the second end of the thulium-doped optical fiber 110a while transmitting the remaining portion.

For example, the second fiber Bragg grating 110c may reflect 99% to 100% and transmits 0% to 1% of the second optical pulse, respectively.

The delay optical fiber 130 delays the first optical pulse transmitted through the second fiber Bragg grating 110c.

Compared to the second optical pulse supplied to the second optical wavelength division multiplexer/demultiplexer 140, a time delay is generated in the first optical pulse when the first optical pulse passes through the delay optical fiber 130.

The time delay may be adjusted by adjusting the length of the delay optical fiber 130.

The delay optical fiber 130 may include a single mode optical fiber.

The second optical wavelength division multiplexer/demultiplexer 140 combines the first optical pulse delayed by the delay optical fiber 130 and the second optical pulse separated and outputted by the first optical wavelength division multiplexer/demultiplexer 120 to output a dual wavelength optical pulse.

The second optical wavelength division multiplexer/demultiplexer 140 has the same structure and operation as the first optical wavelength division multiplexer/demultiplexer 120. Thus, a detailed description will not be given.

The dual wavelength optical pulse outputted from the second optical wavelength division multiplexer/demultiplexer 140 includes the first optical pulse having the first wavelength and the second optical pulse having the second wavelength that are alternately repeated at a constant cycle.

Hereinafter, the operation of the gain switching laser generator 1000 according to the first embodiment of the present invention will be described in detail.

When the optical pump 100 supplies the first optical pulse having the first wavelength (e.g., 1560±30 nm) to the first optical wavelength division multiplexer/demultiplexer 120, the first optical wavelength division multiplexer/demultiplexer 120 introduces the first optical pulse into the first end of the thulium-doped optical fiber 110a via the first fiber Bragg grating 110b.

When the first optical pulse generated by the optical pump 100 passes through the thulium-doped optical fiber 110a, which is a gain medium, an initial spontaneous emission of the second optical pulse having the second wavelength (e.g., 1705±30 nm) occurs. That is, a seed beam of the second optical pulse is generated by the spontaneous emission.

The first optical pulse transmitted through the thulium-doped optical fiber 110a passes through the second fiber Bragg grating 110c and is introduced into the delay optical fiber 130.

The second optical pulse generated by spontaneous emission is reflected by the second fiber Bragg grating 110c and is introduced back into the thulium-doped optical fiber 110a.

The second optical pulse introduced back into and transmitted through the thulium-doped optical fiber 110a is reflected by the first fiber Bragg grating 110b and is introduced back into the thulium-doped optical fiber 110a.

The spontaneous emission of the second optical pulse is induced by the first optical pulse supplied by the optical pump 100, and subsequently, a large amount of stimulated emission of the second optical pulse is also induced by the second optical pulse that goes back and forth between the first fiber Bragg grating 110b and the second fiber Bragg grating 110c. That is, resonance occurs to generate a large amount of second optical pulse.

The large amount of second optical pulse generated by stimulated emission passes through the first fiber Bragg grating 110b and is then introduced into the first optical wavelength division multiplexer/demultiplexer 120.

The first optical wavelength division multiplexer/demultiplexer 120 separates the input second optical pulse and supplies the separated second optical pulse to the second optical wavelength division multiplexer/demultiplexer 140.

The second optical wavelength division multiplexer/demultiplexer 140 then combines the first optical pulse delayed by the delay optical fiber 130 and the second optical pulse separated and outputted by the first optical wavelength division multiplexer/demultiplexer 120 to output a dual wavelength optical pulse.

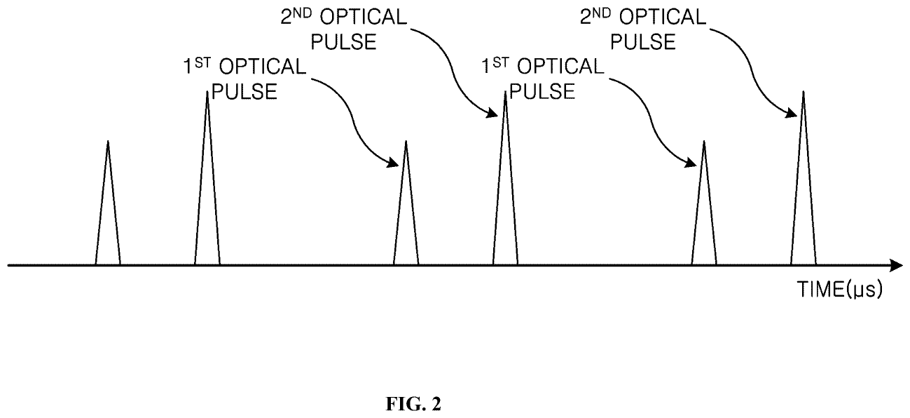

FIG. 2 schematically illustrates a dual wavelength optical pulse outputted by the dual wavelength optical pulse generator according to the first embodiment of the present invention.

Referring to FIG. 2, the dual wavelength optical pulse outputted from the second optical wavelength division multiplexer/demultiplexer 140 includes the first optical pulse and the second optical pulse that are alternately repeated.

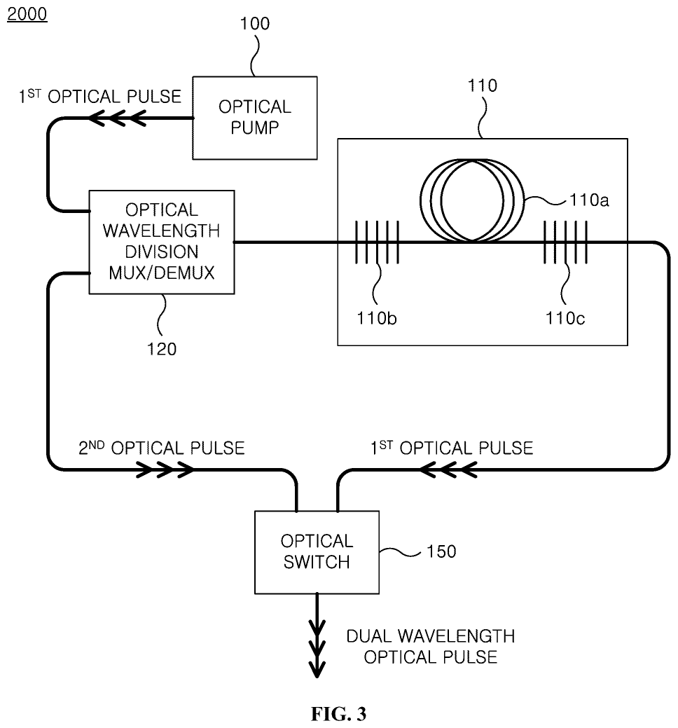

FIG. 3 schematically illustrates a dual wavelength optical pulse generator according to a second embodiment of the present invention.

Referring to FIG. 3, a dual wavelength optical pulse generator 2000 according to the second embodiment of the present invention includes an optical pump 100, an optical resonator 110, an optical wavelength division multiplexer/demultiplexer 120 and an optical switch 150.

The optical pump 100 generates a first optical pulse (e.g., a pulse laser) having a first wavelength and provides the same to the optical wavelength division multiplexer/demultiplexer 120.

The optical pump 100 generates a high-power first optical pulse using an erbium-doped optical fiber, a ytterbium-doped optical fiber, etc.,

Preferably, the range of the first wavelength is 1560±30 nm, but is not limited thereto.

The optical wavelength division multiplexer/demultiplexer 120 provides the first optical pulse generated by the optical pump 100 to a first end of the optical resonator 110, and separates and outputs the second optical pulse generated by the optical resonator 110.

Here, the optical wavelength division multiplexer/demultiplexer 120 is the same as the first optical wavelength division multiplexer/demultiplexer 120 according to the first embodiment described above. Thus, a detailed description will not be given.

The optical resonator 110 generates a second optical pulse having a second wavelength from the first optical pulse generated by the optical pump 100.

Specifically, the optical resonator 110 may include a thulium-doped optical fiber 110a, a first fiber Bragg grating 110b and a second fiber Bragg grating 110c.

The thulium-doped optical fiber 110a generates the second optical pulse having a second wavelength from the first optical pulse generated by the optical pump 100.

The thulium-doped optical fiber 110a generates a second optical pulse having a second wavelength from the first optical pulse having the first wavelength via spontaneous emission and subsequent stimulated emission. That is, the thulium-doped optical fiber 110a acts as a gain medium of a resonator for generating the second optical pulse, which will be described later.

Preferably, the range of the second wavelength is 1705±30 nm, but is not limited thereto.

The first fiber Bragg grating 110b is provided between the optical wavelength division multiplexer/demultiplexer 120 and the first end of the thulium-doped optical fiber 110a to partially reflect the second optical pulse outputted from the first end of the thulium-doped optical fiber 110a. The first fiber Bragg grating 110b functions as a mirror that constitutes the optical resonator 110.

The first fiber Bragg grating 110b may reflect a portion of the second optical pulse outputted from the first end of the thulium-doped optical fiber 110a while transmitting the remaining portion.

For example, the first fiber Bragg grating 110b may reflect 60% to 90% and transmits 10% to 40% of the second optical pulse, respectively.

The second fiber Bragg grating 110c is provided at a second end of the thulium-doped optical fiber 110a and transmits the first optical pulse outputted from the second end of the thulium-doped optical fiber 110a and also reflects the second optical pulse.

Specifically, the second fiber Bragg grating 110c may transmit the entirety of the first optical pulse remaining after being used as a pump beam. The second fiber Bragg grating 110c functions as a mirror constituting the optical resonator 110.

The second fiber Bragg grating 110c may reflect a portion of the second optical pulse outputted from the second end of the thulium-doped optical fiber 110a while transmitting the remaining portion.

For example, the second fiber Bragg grating 110c may reflect 99% to 100% and transmits 0% to 1% of the second optical pulse, respectively.

The optical switch 150 selectively outputs the first optical pulse passed through the second fiber Bragg grating 110c and the second optical pulse separated and outputted by the wavelength division multiplexer/demultiplexer 120 according to the user's selection.

Hereinafter, the operation of the gain switching laser generator 2000 according to the second embodiment of the present invention will be described in detail.

When the optical pump 100 supplies the first optical pulse having the first wavelength (e.g., 1560±30 nm) to the optical wavelength division multiplexer/demultiplexer 120, the optical wavelength division multiplexer/demultiplexer 120 introduces the first optical pulse into the first end of the thulium-doped optical fiber 110a via the first fiber Bragg grating 110b.

When the first optical pulse generated by the optical pump 100 passes through the thulium-doped optical fiber 110a, which is a gain medium, an initial spontaneous emission of the second optical pulse having the second wavelength (e.g., 1705±30 nm) occurs. That is, a seed beam of the second optical pulse is generated by the spontaneous emission.

The first optical pulse transmitted through the thulium-doped optical fiber 110a passes through the second fiber Bragg grating 110c and is introduced into the optical switch 150.

The second optical pulse generated by spontaneous emission is reflected by the second fiber Bragg grating 110c and is introduced back into the thulium-doped optical fiber 110a. The second optical pulse introduced back into and transmitted through the thulium-doped optical fiber 110a is reflected by the first fiber Bragg grating 110b and is introduced back into the thulium-doped optical fiber 110a.

The spontaneous emission of the second optical pulse is induced by the first optical pulse supplied by the optical pump 100, and subsequently, a large amount of stimulated emission of the second optical pulse is also induced by the second optical pulse that goes back and forth between the first fiber Bragg grating 110b and the second fiber Bragg grating 110c. That is, resonance occurs to generate a large amount of second optical pulse.

The large amount of second optical pulse generated by stimulated emission passes through the first fiber Bragg grating 110b and is then introduced into the optical wavelength division multiplexer/demultiplexer 120.

The optical wavelength division multiplexer/demultiplexer 120 separates the introduced second optical pulse and supplies the same to the optical switch 150.

The optical switch 150 selectively outputs the first optical pulse passed through the second fiber Bragg grating 110c and the second optical pulse separated and outputted by the first optical wavelength division multiplexer/demultiplexer 120 according to the user's selection.

For example, the optical switch 150 may output either the first optical pulse or the second optical pulse, depending on the user's selection.

The dual wavelength optical pulse generator according to the present invention has the following advantages.

(1) The dual wavelength optical pulse generator according to the present invention is advantageous in that a high-power 1700 nm band laser at low cost using thulium-doped optical fiber may be generated.

(2) The dual wavelength optical pulse generator according to the present invention is advantageous in that an optical pulse having dual wavelengths of 1500 nm band and 1700 nm band at low cost may be generated.

(3) The dual wavelength optical pulse generator according to the present invention is advantageous in that an optical pulse of 1500 nm band and 1700 nm band may be generated with a single device and generated optical pulse may be selectively used.

What is claimed is:

1. A dual wavelength optical pulse generator, comprising:
an optical pump generating a first optical pulse having a first wavelength;
an optical resonator generating a second optical pulse having a second wavelength from the first optical pulse generated by the optical pump;
a first optical wavelength division multiplexer/demultiplexer supplying the first optical pulse generated by the optical pump to a first end of the optical resonator and separating and outputting the second optical pulse generated by the optical resonator;
a delay optical fiber delaying the first optical pulse passed through the optical resonator; and
a second optical wavelength division multiplexer/demultiplexer combining: the first optical pulse delayed by the delay optical fiber; and the second optical pulse separated and outputted by the first optical wavelength division multiplexer/demultiplexer to output a dual wavelength optical pulse,
wherein the optical resonator comprises:
a thulium-doped optical fiber generating the second optical pulse from the first optical pulse;
a first fiber Bragg grating provided at a first end of the thulium-doped optical fiber and reflecting the second optical pulse outputted from the first end of the thulium-doped optical fiber; and
a second fiber Bragg grating provided at a second end of the thulium-doped optical fiber, the second fiber Bragg grating transmitting the first optical pulse therethrough and reflecting the second optical pulse outputted from the second end of the thulium-doped optical fiber.

2. The dual wavelength optical pulse generator of claim 1, wherein ranges of the first wavelength and the second wavelength are 1560±30 nm and 1705±30 nm, respectively.

3. The dual wavelength optical pulse generator of claim 1, wherein the first fiber Bragg grating reflects 60% to 90% and transmits 10% to 40% of the second optical pulse, respectively.

4. The dual wavelength optical pulse generator of claim 1, wherein the second fiber Bragg grating reflects 99% to 100% and transmits 0% to 1% of the second optical pulse.

5. The dual wavelength optical pulse generator of claim 1, wherein the delay optical fiber comprises a single mode optical fiber.

6. A dual wavelength optical pulse generator, comprising:
an optical pump generating a first optical pulse having a first wavelength;
an optical resonator generating a second optical pulse having a second wavelength from the first optical pulse generated by the optical pump;
a optical wavelength division multiplexer/demultiplexer supplying the first optical pulse generated by the optical pump to a first end of the optical resonator and separating and outputting the second optical pulse generated by the optical resonator;
a delay optical fiber delaying the first optical pulse passed through the optical resonator; and
an optical switch selectively outputting: the first optical pulse passed through the optical resonator; and the second optical pulse separated and outputted by the optical wavelength division multiplexer/demultiplexer,
wherein the optical resonator comprises:
a thulium-doped optical fiber generating the second optical pulse from the first optical pulse;
a first fiber Bragg grating provided at a first end of the thulium-doped optical fiber and reflecting the second optical pulse outputted from the first end of the thulium-doped optical fiber; and
a second fiber Bragg grating provided at a second end of the thulium-doped optical fiber, the second fiber Bragg grating transmitting the first optical pulse therethrough and reflecting the second optical pulse outputted from the second end of the thulium-doped optical fiber.

7. The dual wavelength optical pulse generator of claim 6, wherein ranges of the first wavelength and the second wavelength are 1560±30 nm and 1705±30 nm, respectively.

8. The dual wavelength optical pulse generator of claim 6, wherein the first fiber Bragg grating reflects 60% to 90% and transmits 10% to 40% of the second optical pulse, respectively.

9. The dual wavelength optical pulse generator of claim 6, wherein the second fiber Bragg grating reflects 99% to 100% and transmits 0% to 1% of the second optical pulse.

10. The dual wavelength optical pulse generator of claim 6, wherein the delay optical fiber comprises a single mode optical fiber.
